(19) United States
(12) Patent Application Publication
(10) Pub. No.: US 2019/0206527 A1
(43) Pub. Date: Jul. 4, 2019

(54) REGISTER FOR EXAMINATIONS WITH CONTRAST AGENT

(71) Applicants: Siemens Healthcare GmbH, Erlangen (DE); Siemens Medical Solutions USA, Inc., Malvern, PA (US)

(72) Inventors: Christine Boehm, Erlangen (DE); Robert Krieg, Nuernberg (DE); Richard Frank, Alexandria, VA (US); Lars Lauer, Neunkirchen (DE)

(73) Assignees: Siemens Healthcare GmbH, Erlangen (DE); Siemens Medical Solutions USA, Inc., Malvern, PA (US)

(21) Appl. No.: 15/858,196

(22) Filed: Dec. 29, 2017

(51) Int. Cl. G16H 10/60; A61B 5/055; A61B 5/00; A61B 6/03; G16H 30/40; G01R 33/56

(52) U.S. Cl. CPC G16H 10/60; A61B 5/055; A61B 5/7267; G01R 33/5608; G16H 30/40; G01R 33/5601; A61B 6/037

(57) ABSTRACT

In a method and a computer for identifying a group of persons, from a number of persons, to whom at least one contrast agent has been applied during an examination which was carried out in an imaging system with which medical images of each of the number of persons were produced, existing examination data provided for each of the number of persons are used to determine to which of those persons the at least one contrast agent was applied. The persons to whom at least one contrast agent was applied during the examination are identified as a group of persons. For each person in the group, person-specific contrast agent data are determined that include at least identification information identifying the person to whom the at least one contrast agent was applied, and contrast agent information identifying the fact that the at least one contrast agent was applied. A designation of each person in the group of persons is stored with that person's corresponding person-specific contrast agent data in a register.

### BACKGROUND OF THE INVENTION

### Field of the Invention

The present invention concerns a method for identifying a group of persons, from a number of persons, to whom at least one contrast agent has been applied during an examination that was carried out in an imaging system. The invention furthermore relates to a corresponding computer, and a non-transitory data storage medium with program code, designed to identify such a group of persons to whom at least one contrast agent has been applied.

### Description of the Prior Art

Magnetic resonance imaging (MRI) is a non-invasive modality that is commonly used to obtain medical images of a person during an examination. The generated MR images can show pathological or other physiological alterations of the examined tissue. In some cases, contrast agents are used to improve the contrast between different tissues in the examined body.

The use of contrast agents is not limited to MRI systems. Contrast agents are also used in computed tomography and conventional X-ray imaging, as well as in PET (Positron Emission Tomography) systems.

Currently, discussions are ongoing as to whether a deposition of the contrast agent in the human body, especially in the brain, occurs. In this context, the question is asked as to whether registries should be set up that are to be filled, by radiologists, radiographers, or other medically trained personnel involved in such examinations, with patient-specific and examination-specific data for every contrast agent-based examination in an MRI system. This registry may also accumulate patient-specific and examination-specific data for a group of patients who did not receive a contrast agent.

Manual fulfilment of such reporting requirements would adversely affect the MR workflow and increase the overhead cost. Furthermore, it would be nearly impossible to do this work for old examinations carried out over a longer period such as the last several years.

### SUMMARY OF THE INVENTION

Accordingly, a need exists to provide an automated procedure by which it is determined whether a person or patient has received a contrast agent during an examination, and that accesses attributes of the acquired image that enable the identification of patient groups.

According to a first aspect of the invention, a method is provided for identifying a group of persons, from a number of persons, to whom least one contrast agent has been applied during an examination which was carried out with an imaging system that generated medical images of each of the number of persons. The number of persons from whom images were obtained with the imaging system includes patients to whom no contrast agent was administered as well as patients who received a contrast agent for the examination. The method includes determining, based on existing examination data provided for each of the number of persons, which persons in the number of persons, to whom at least one contrast agent has been applied. The persons to whom a contrast agent was administered during the examination are identified as the aforementioned group of persons. For each person of this group, person-specific contrast agent data are determined that include at least identification information identifying the person to whom the at least one contrast agent was applied. The person-specific contrast agent data may furthermore include contrast agent information identifying the fact that the at least one contrast agent was applied. Each person of the determined first group is then stored with that person's corresponding person-specific contrast agent data in a register.

When an examination has been carried out, the existing examination data provided for each person include data that allow the determination of the person-specific contrast agent data, which specify that a contrast agent has been applied and which identify the corresponding person. All the generated medical images are stored together with the identity of the examined person. Furthermore, either the generated medical images themselves or the additional data stored with the medical images, i.e., the attributes attached to the images, allow an identification of the examinations in which contrast agents have been used.

Accordingly, it is possible to identify the persons to whom a contrast agent has been applied by processing the examination data available to each examined person. It is possible to make the file anonymous by stripping from the file only the patient- or person-specific identifier. Furthermore, it is possible to assign an unambiguous alphanumeric or digital identifier to enable linking data from the same patient/person obtained longitudinally, that is, over a period of time, before or after the storing of this file.

For determining to which persons among the number of persons the at least one contrast agent was applied, the generated medical images for each of the number of persons can be analyzed in order to detect contrast enhanced images that were acquired during or after administration of the at least one contrast agent. The group of persons is then identified based on the detected contrast enhanced images. Thus the medical images that were previously generated are used to determine whether a contrast agent was applied. These images differ from other images by the readily-apparent contrast generated in the image, or by the contrast obtained for different tissues. Accordingly by post-processing the generated images, it is possible to determine whether a contrast agent was applied or not.

Furthermore, it is possible to determine the identification information identifying the person to whom the at least one contrast agent has been applied from attributes attached to each of the contrast enhanced images.

Such medical images customarily contain attributes such as a header in which additional information relating to the images are stored. This additional information includes the full name of the examined person and further information describing details about the procedure by which the image was obtained. One known data structure used for medical images is the DICOM standard, so that is possible to deduce the identification information from the DICOM header provided for each of the generated medical images. Furthermore, it is possible to deduce the contrast agent information from the DICOM header. The DICOM header can contain information about the use imaging protocol or other indications. These indications often contain names that allow the fact that a contrast agent has been applied to be deduced from this data. It is possible to retain DICOM header information despite stripping from the file only the patient-/person-specific identifier, whether or not an unambiguous alphanumeric or digital identifier is assigned in place of the patient-specific identifier.

For determining to which of the number of persons the at least one contrast agent has been applied, it is also possible to process the imaging protocol data stored in connection with the medical images that are part of the existing examination data. The imaging protocol data include at least information about an imaging protocol used for generating the medical images generated for the corresponding person. Accordingly, it is possible to identify the group of persons by searching for imaging protocol data that have an indication that a contrast agent was applied for generating the medical images. The protocol names often contain keywords in the protocol name or a dedicated protocol or patient registry parameter. Using these indications, it is possible to determine whether a contrast agent was applied or not.

Furthermore, it is possible to determine to which of the number of persons the contrast agent has been applied by processing the medical images in a computer that operates with artificial intelligence. This computer with artificial intelligence may be trained with training medical images that contain non-contrast enhanced training images, which were acquired without administration of the contrast agent, and contrast enhanced training images, which were acquired after or during administration of the contrast agent. For each of the training medical images, it is known whether they were acquired with our without administration of the contrast agent. In such a case a computer with artificial intelligence, such as neural networks, provides very precise and accurate results, as it need only make a digital decision of yes or no. By processing the generated medical images in the processing device having artificial intelligence, which was trained with the training medical images, it can be determined with a high precision for each of the medical images whether it is a contrast enhanced image or non-contrast enhanced image, using the knowledge obtained with the training medical images.

This option has the benefit that a false reporting in the attributes generated by a person involved in the examination can be avoided. For example, if a contrast enhanced imaging protocol is used without a contrast agent, the above mentioned use of the intelligence can determine with a high precision whether contrast agents were actually used in an obtained medical image or not.

A further option is to identify at least one anatomical structure in the medical images by determining an image intensity in the identified anatomical structure. The group of persons can then be identified by taking into account the identified at least one anatomical structure with the corresponding image intensity.

Especially when the imaging system is an MRI system and the generated medical images are MR images it is possible to precisely predict which image contrast can be expected in which anatomical structure, when further imaging parameters such as echo time TE, repetition time TR, or inversion time TI are known. When the image intensity in the medical image then differs from the expected image intensity that is expected without the use of contrast agent and, e.g., if the difference is not within a certain threshold it can be determined that a contrast agent has been applied. By means of these imaging attributes, especially when the imaging system in an MRI system, it is possible to characterize the persons in this group of persons for the purpose of classifying them into sub-groups according to person-specific findings. The characteristics defining persons in these sub-groups of the group of persons could then be used to identify, from the original number of persons, a second set of sub-groups, similar to the first in characteristics (i.e., a “cohort”), but without contrast.

Furthermore, the existing examination data stored in connection with the medical images sometimes contain information which contrast agent has been applied. Accordingly, it is possible to determine from the existing examination data which contrast agent has been applied wherein the applied contrast agent and the information about the applied contrast agent can be stored in the person-specific contrast agent data and can thus be stored in the register together with the identity of the person. It is possible to retain information regarding which contrast agent has been applied, despite de-identification by stripping from the file only the patient-specific identifier. This holds true whether or not an unambiguous alphanumeric or digital identifier is assigned in place of the patient-specific identifier.

Furthermore, the corresponding computer configured to identify the group of persons from a number of persons is provided with a memory and at least one processor, wherein the memory contains instructions executable by the at least one processor.

The present invention also encompasses a non-transitory, computer-readable data storage medium encoded with programming instructions that, when the storage medium is loaded into a computer, cause the computer to implement any or all embodiments of the method according to the invention, as described above.

Is to be understood that the features mentioned above and features explained below can be used not only in the respective combinations indicated, but also in other combinations or in isolation without departing from the scope of the present invention. Features of the above mentioned aspects and embodiments described below may be combined with each other in other embodiments unless explicitly mentioned otherwise.

### BRIEF DESCRIPTION OF THE DRAWINGS

FIG. 1 is a schematic illustration of an imaging system embodied as an MRI system, with which a contrast agent is applied to a person, wherein a computer automatically determines from the generated medical images whether a contrast agent was applied or not and stores the corresponding information in a register.

FIG. 2 is a schematic illustration of how a computer with artificial intelligence, which was trained with training medical images, can be used to automatically determine whether a contrast agent was applied in a generated medical image.

FIG. 3 is a schematic illustration of a medical image that contains further attributes that allow an identity of the examined person and/or the use of the contrast agent to be determined.

FIG. 4 is a flowchart carried out by the computer shown in FIG. 1 in order to implement the method.

### DESCRIPTION OF THE PREFERRED EMBODIMENTS

In the following, embodiments of the invention will be described in detail with reference to the accompanying drawings. It is to be understood that the following description of embodiments is not to be taken in a limiting sense. The scope of the invention is not intended to be limited by the embodiments described hereinafter or by the drawings, which are illustrative only.

The drawings are to be regarded as being schematic representations, and elements illustrated in the drawings are not necessarily shown to scale. Rather, the various elements are represented such that their function and general purpose becomes apparent to those skilled in the art. Any connection or coupling between functional blocks, devices, components, of physical or functional units shown in the drawings and described hereinafter may also be implemented in an indirect connection or coupling. A coupling between components may be established by a wired or wireless connection. Functional blocks may be implemented in hardware, software, firmware, or a combination thereof.

As will be explained below, a method is provided which identifies the persons, from a number of persons, to whom a contrast agent has been applied using existing examination data provided for each of the examined persons. All the data needed for identifying a person and identifying the fact whether contrast agent has been applied is available at the stored medical images or at the imaging system such as an MRI system. When a person or patient is registered before the examination the identity is known and this kind of data is part of the examination data of the examined person. An automated data feed from the imaging system to a register is possible and could hence free the radiologist or other persons involved during the examination from this administrative work.

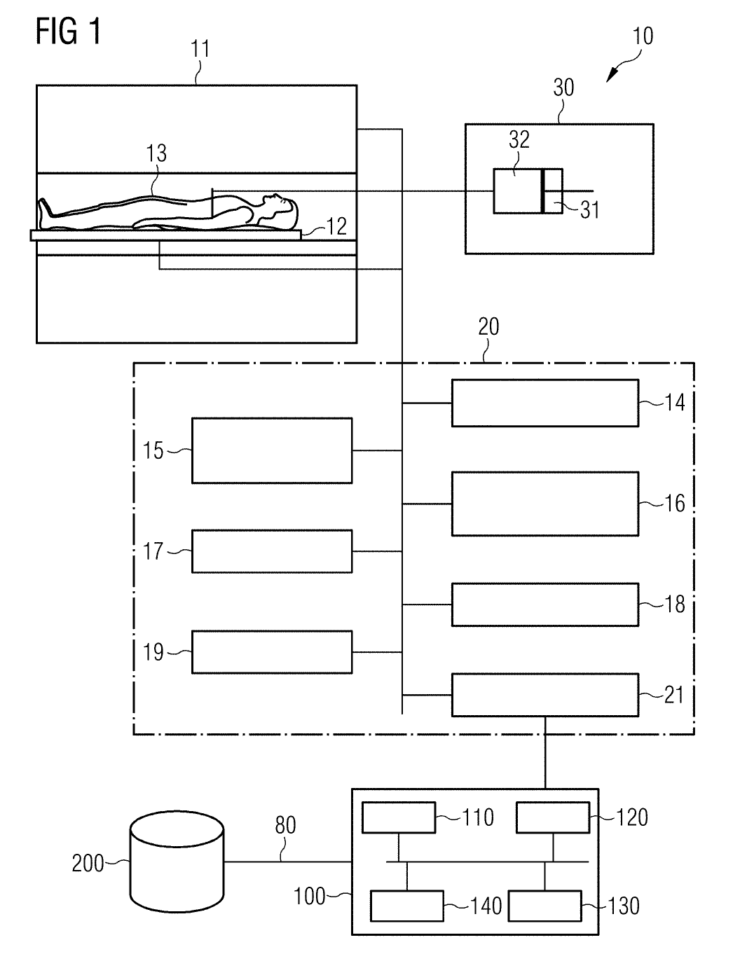

FIG. 1 schematically shows an MRI system 10 that is connected to a computer 100, which will deduce from the existing examination data provided for a person, whether a contrast agent has been applied. Designations of the persons for whom it has been determined that a contrast agent has been applied are stored together with additional information in a register 200.

In the following the use of the MRI system is briefly explained in more detail. The MRI system 10 has a scanner with a basic field magnet 11 that generates the polarization field BO, wherein a person 13 is located on a support 12 and is positioned within the magnet 11, where images of the person 13 can be generated. Furthermore, a contrast agent injector 30 is provided with a container 31 filled with a contrast agent 32 that can be applied before or during an examination at predefined moments in time in order to obtain a certain effect due to the applied contrast agent in the generated MR images. The MRI system 10 furthermore has receiving coils (not shown) in order to detect the MR signal. By applying RF pulses and by applying magnetic field gradients, the generated magnetization can be excited and detected by the receiving coils. How an MR image is obtained from the detected MR signal is known to those skilled in the art, and need not be explained in further detail herein.

The MRI system 10 furthermore has a control computer 20 that controls the operation of the MRI system 10. The control computer 20 has a gradient controller 15 for controlling the gradients applied during the imaging process. Furthermore, a RF controller 14 for the RF pulse to be generated is provided. An imaging sequence controller 16 controls the sequence of the applied magnetic field gradients and the RF pulses and the signal detection and thus also controls controllers 14 and 15. An input unit 17 is provided that is operable by a user of the MRI system 10 to control the system 10, and a display 18 is provided at which the MR images can be displayed. A processor 19 is provided for processing and controlling the different entities of the controller 20. Furthermore, a memory 21 can be provided where program modules can be stored that are needed for the operation of the MRI system, and where the MR images and the further information obtained during an examination may be stored.

The computer 100 is shown as being connected to the MRI system 10, especially to the memory where the MR images may be stored. The computer 100 has a memory 110 and at least one processor 120. The processor 120 is responsible for the operation of the computer 100 and carries out instructions stored on the memory 110. The memory may include a read-only memory, random access memory, a mask storage, a hard disk or the like. The memory 110 can furthermore include suitable program code to be executed by the processor 120 so as to implement the functionalities in which the computer 100 is involved, and which are described in further detail below. The computer 100 can include further functional entities which are not shown for of clarity, such as a human machine interface.

The computer 100 has a display 130, an interface 140 for the communication with the MRI system and for the communication with the register 200 where all the persons who are identified as persons to which a contrast agent has been applied, are stored. The register 200 can be a separate database or can be incorporated into the processing entity 100 or may be even incorporated into the MRI system 10. A connection 80 is provided for connecting the register 200 to the processing entity wherein the connection 80 may include a wide area network such as the Internet or a local area network. In the system of FIG. 1, the components of the computer 100 are shown as being separated from the components of the MRI system. It is possible for the functionality provided by the computer 100 to be incorporated into the control computer 20, here the processor 19 and the memory 21.

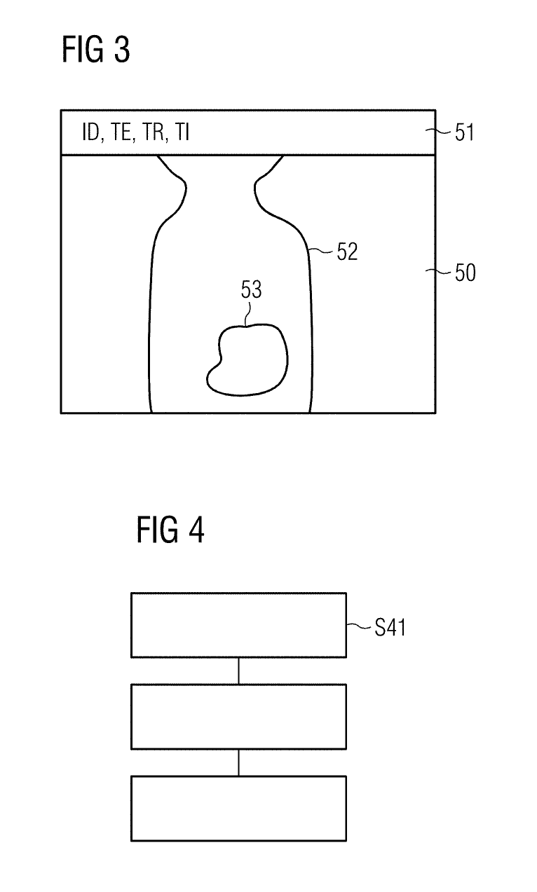

The computer 100 determines, for each of the persons which underwent an examination in the MRI system, whether a contrast agent was applied. To this end the computer 100 first determines the identity for each of the persons. The identity of the person may be directly deduced from the patient data provided in the MRI system when the patient has been registered before the examination is carried out. Furthermore, the identification data identifying the person may be deduced from the generated image data itself. As shown in FIG. 3, an MR image 50 has, in addition to the imaging data, additional attributes that are shown symbolically as being stored in a header 51 of the image. This header 51 can contain the data identifying the person. The header 51 can furthermore contain information about the imaging protocol or imaging parameters used for generating the image. In case of an MRI system, the imaging parameters such as echo time TE, repetition time TR or inversion time TI may be provided in the header. In the medical field a kind of standardised header is used which is known as the DICOM header and which allows the exchange of data by different systems. Accordingly, the identification information can be deduced from the DICOM header 51 or from the registration data when the patient is registered at the imaging system. Furthermore, it is possible to use the attributes stored in the header 51 to determine whether a contrast agent has been applied. The header 51 can contain the name of the used imaging protocol or can contain certain keywords in the protocol name or a certain protocol parameter which indicates that a contrast agent has been applied. Furthermore, the name of the contrast agent may be stored in this context. Accordingly, by processing these attributes provided for each image it may be possible to not only determine the identification information identifying the person but also to identify the contrast agent information identifying the fact that the contrast agent has been applied.

A further option for the identification of the contrast agent information is to post-process the imaging data.

As an example, the image itself and certain anatomical structures may be identified. In the example of FIG. 3, the person 52 has an anatomical structure 53. By image post-processing techniques it is possible to identify an anatomical structure such as a certain organ or any other anatomical structure. When the anatomical structure is identified and the image intensity in the anatomical structure 53 is determined from the image, it is possible to determine whether a contrast agent has been applied when further knowledge about the image is provided, such as the imaging protocol or the imaging parameters. When some of the imaging parameters are known it is possible to theoretically compute the expected image intensity in the image. If the image intensity does not correspond to the determined value and differs from the explicit value by more than a certain extent, it can be determined that a contrast agent has been applied. Furthermore, it is known how the contrast agents influence the T1 or T2 times in the MR image. Based on this knowledge it is also possible to directly determine from the image intensity and the identified anatomical region whether a contrast agent has been applied by comparing the detected image intensity to a calculated image intensity which has been calculated based on the assumption that a contrast agent has been applied or not. If the expected intensity is within a range similar to the actually detected intensity, the processing entity can deduce that a contrast agent has been applied in an image.

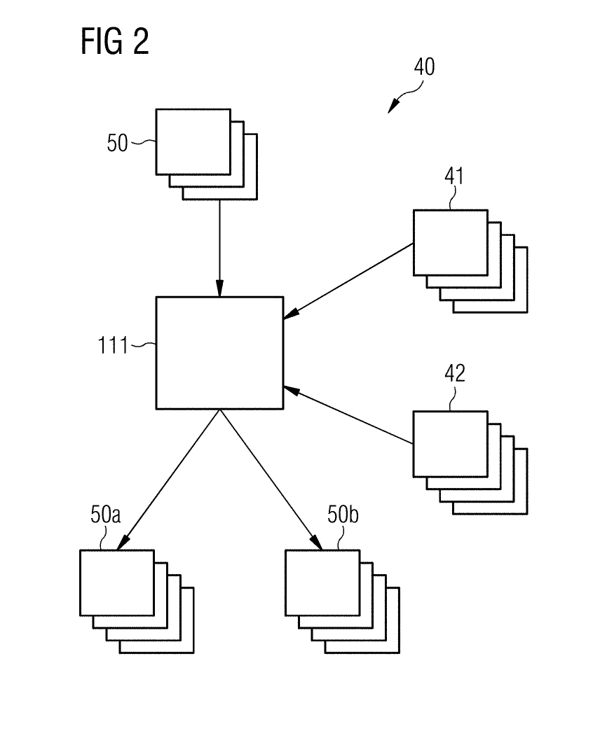

FIG. 2 shows a further option that can be used to determine whether a contrast agent has been applied or not. As this is a digital decision with yes or no, this question can be answered with a very high precision by a system having artificial intelligence. Accordingly, the processor 110 can be a processing device having artificial intelligence 111 as symbolized in FIG. 2. This processing device with artificial intelligence 111 can be trained with training medical images 41 and 42. These training medical images contain non-contrast enhanced training images 41 and contrast enhanced training images 42. The images 41 were acquired without administration of the contrast agent and this is known to the device 111 and the contrast enhanced training images 42 were acquired after or during administration of the contrast agent. The device can learn from the information how the contrast is in a contrast enhanced image and how the contrast is in a non-contrast enhanced image and can build up knowledge which makes it possible to process the newly generated images such as the medical images 50 generated by the MRI system shown in FIG. 1 for which it is no known whether a contrast agent has been applied or not. When these medical images for which it is not known whether a contrast agent has been applied are fed to the device 111 the device 111 can use the trained knowledge in order to differentiate the non-contrast enhanced images 50a and the contrast enhanced images 50b. For the contrast enhanced images 50b the corresponding identification information can be determined for identifying the corresponding person. The person-specific contrast agent data are stored in register 200. The person-specific contrast agent data indicate the identity of the person and the fact that a contrast agent has been applied. Preferably also the kind of contrast agent is identified, if possible.

FIG. 4 summarizes the steps carried out by the processing entity 100 shown in FIG. 1.

In step S41 the existing examination data of the examined persons are used to identify the group of persons to whom a contrast agent was applied. In step S42 it is determined for each of the persons of the first group, what the identity of the person is and the fact is determined that a contrast agent has been applied, preferably also the fact which contrast agent was applied. In step S43, for each of the persons, the person-specific contrast agent data are stored in register 200.

As discussed above, different options exist for determining whether a contrast agent has been applied. The option discussed above in connection with FIG. 2 is attractive as far as false reporting resulting from an accidental use of contrast based imaging protocols in the absence of a contrast agent can be excluded.

The different options discussed in connection with FIGS. 2 and 3 may also be deployed centrally, for example, in a PACS system of the radiology department or the clinic bypassing any images stored in the system so that contrast agent based examinations can be identified. Accordingly, even retrospective reporting requirements can be fulfilled automatically by analyzing archives storing older images.

In the above discussed example the invention was discussed in connection with MRI systems where often gadolinium based contrast agents are used. However, the invention is not limited to MRI systems and MRI-specific contrast agents. In a computer tomograph lowdine based contrast agents are used or in conventional X-ray contrast agents are used. Furthermore, various PET and SPECT tracers are used which all influence the image appearance. Accordingly, all the images generated by these different imaging systems can be processed as discussed above. As all the reported relevant data such as the date, time, the patient name, age, gender et cetera are part of the DICOM header, a standardised input source already exists allowing a widespread use of the solution mentioned above.

Although modifications and changes may be suggested by those skilled in the art, it is the intention of the Applicant to embody within the patent warranted hereon all changes and modifications as reasonably and properly come within the scope of the Applicant's contribution to the art.

1. A method for identifying a group of persons to whom a contrast agent was administered, comprising:
providing a computer with existing examination data acquired from patients who underwent an examination in a medical imaging system during which at least one medical image was generated for each person, at least some of said examinations with said imaging system requiring administration of a contrast agent to at least some of the persons represented in the existing examination data;
in said computer, analyzing said examination data in order to identify said persons to whom a contrast agent was administered, and collecting the identified persons as a group of persons;
in said computer, for each person in said group of persons, determining patient-specific contrast data comprising at least identification information that identifies the person to whom the contrast agent was administered, and contrast agent information identifying that the contrast agent was administered; and
in a memory in communication with said computer, storing each person in said group of persons together with that person's corresponding patient-specific contrast agent data, and thereby forming a register of the persons in said group of persons in said memory.

2. A method as claimed in claim 1 wherein said examination data, for the persons in said group, includes contrast-enhanced images for the persons in said group of persons to whom the contrast agent was administered, and, in said computer, placing a person, represented in said examination data, in said group of persons, by detecting that a contrast-enhanced image was generated for that person.

3. A method as claimed in claim 2 comprising determining said identification information from attributes associated with the respective contrast enhanced images.

4. A method as claimed in claim 1 comprising placing persons, represented in said examination data, in said group of persons by analyzing, in said computer, imaging protocol data for the respective examination with the medical imaging system conducted on the respective persons, and placing a person in said group of persons when the respective imaging protocol data indicate that a contrast agent was applied in the examination of that respective person.

5. A method as claimed in claim 4 comprising, in said computer, searching said image protocol data using a search term selected from the group consisting of a protocol name that indicates that contrast agent was applied during the respective examination, and a predetermined expression that is present in a protocol name that indicates that contrast agent was administered during the respective examination.

6. A method as claimed in claim 1 comprising placing persons, represented in said examination data, in said group of persons by artificial intelligence in said computer that is trained with training medical images, comprising non-contrast-enhanced training images that were acquired without administration of a contrast agent, and contrast-enhanced training images that were acquired after or during administration of a contrast agent, together with knowledge, for each of the training medical images, as to whether a respective training medical image was acquired with or without administering the contrast agent, and processing the medical images represented in the examination data using said trained artificial intelligence in order to identify whether a medical image in the examination data was or was not acquired with administration of a contrast agent.

7. A method as claimed in claim 1 comprising placing persons, represented in said examination data, in said group of persons by, in said computer, identifying at least one anatomical structure in the medical image associated with a respective person, and determining an image intensity in the identified anatomical structure and, from said image intensity, determining whether a contrast agent was applied in order to generate the medical image of the identified anatomical structure.

8. A method as claimed in claim 1 comprising providing said computer with examination data acquired from a magnetic resonance imaging system, wherein the generated medical images are magnetic resonance images.

9. A method as claimed in claim 8 comprising placing persons, represented in said examination data, in said group of persons by analyzing, in said computer, imaging parameters in the respective medical images of the persons represented in said examination data, with regard to at least one of an echo time in the respective magnetic resonance images, a repetition time used to generate the respective magnetic resonance images, and an inversion time used to generate the respective magnetic resonance images.

10. A method as claimed in claim 1 wherein each of said medical images has a DICOM header, and determining said identification information by analyzing said DICOM header in said computer.

11. A method as claimed in claim 1 comprising providing said computer, in said existing examination data, with information that designates which contrast agent was applied during each examination, and determining, in said computer, from said existing examination data which contrast agent was applied to each patient, and storing a designation of the applied contrast agent with the person-specific contrast agent data.

12. A computer for identifying a group of persons to whom a contrast agent was administered, comprising:
a processor;
an input interface that provides said processor with existing examination data acquired from patients who underwent an examination in a medical imaging system during which at least one medical image was generated for each person, at least some of said examinations with said imaging system requiring administration of a contrast agent to at least some of the persons represented in the existing examination data;
said processor being configured to analyze said examination data in order to identify said persons to whom a contrast agent was administered, and collecting the identified persons as a group of persons;
said processor being configured for each person in said group of persons, to determine patient-specific contrast data comprising at least identification information that identifies the person to whom the contrast agent was administered, and contrast agent information identifying that the contrast agent was administered;
a memory in communication with said processor; and
said processor being configured to store each person in said group of persons together with that person's corresponding patient-specific contrast agent data in said memory, and thereby form a register of the persons in said group of persons in said memory.

13. A non-transitory, computer-readable data storage medium encoded with programming instructions, said storage medium being loaded into a computer and said programming instructions causing said computer to:
receive existing examination data acquired from patients who underwent an examination in a medical imaging system during which at least one medical image was generated for each person, at least some of said examinations with said imaging system requiring administration of a contrast agent to at least some of the persons represented in the existing examination data;
analyze said examination data in order to identify said persons to whom a contrast agent was administered, and collecting the identified persons as a group of persons;
for each person in said group of persons, determine patient-specific contrast data comprising at least identification information that identifies the person to whom the contrast agent was administered, and contrast agent information identifying that the contrast agent was administered; and
in a memory in communication with said computer, store each person in said group of persons together with that person's corresponding patient-specific contrast agent data, and thereby form a register of the persons in said group of persons in said memory.
